SEQUENCE LISTING (4 sequences)

SEQ ID NO: 1 - protein, 20 aa, Artificial Sequence
  Synthetic Tn-glycopeptide
VPSTPPTPSPSTPPTPSPSC

SEQ ID NO: 2 - protein, 8 aa, Artificial Sequence
  Synthetic Tn-glycopeptide after cleavage with endopeptidase
VPSTPPTP

SEQ ID NO: 3 - protein, 12 aa, Artificial Sequence
  Synthetic Tn-glycopeptide after cleavage with endopeptidase
SPSTPPTPSPSC

SEQ ID NO: 4 - protein, 16 aa, Homo sapiens
TVENQQGQDIDDNWVK

(19) United States
(12) Patent Application Publication
(10) Pub. No.: US 2022/0299522 A1
(43) Pub. Date: Sep. 22, 2022

(54) COMPOSITIONS AND METHODS FOR IDENTIFYING O-LINKED GLYCOSYLATION SITES IN PROTEINS

(71) Applicant: THE JOHNS HOPKINS UNIVERSITY, Baltimore, MD (US)

(72) Inventors: Hui Zhang, Ellicott City, MD (US); Weiming Yang, Baltimore, MD (US)

(21) Appl. No.: 17/638,917

(22) PCT Filed: Aug. 26, 2020
(86) PCT No.: PCT/US2020/047945

§ 371 Date: Feb. 28, 2022

Related U.S. Application Data
(60) Provisional application No. 62/891,497, filed on Aug. 26, 2019.

(51) Int. Cl. G01N 33/68; C12N 9/10; C12N 9/76; C12P 19/18; C12P 21/00; C12P 21/06

(52) U.S. Cl. CPC G01N 33/6848; C12N 9/1051; C12N 9/6427; C12P 19/18; C12P 21/005; C12P 21/06; C12Y 204/01; C12Y 304/21004

(57) ABSTRACT

The present invention relates to the field of protein post-translational modification. More specifically, the present invention provides compositions and methods useful for identifying O-linked glycosylation sites in proteins. In one embodiment, the present invention provides a method for identifying O-linked glycosylation sites of Tn antigen in proteins comprising the steps of (a) digesting proteins present in a sample into peptides; (b) enriching for Tn-glycopeptides; (c) conjugating Tn-glycopeptides to solid phase; (d) labeling Tn using the glycosyltransferse enzyme C1GalT1 and a labeled uridine diphosphate galactose (UDP-Gal) substrate to produce labeled Tn-glycopeptides; (e) releasing the labeled Tn-glycopeptides from the solid-phase using an endopeptidase that cleaves peptides at the N-terminus of O-linked glycans at serine or threonine residues; and (f) mapping O-linked glycosylation sites of Tn antigen using liquid chromatography-mass spectrometry.

### CROSS-REFERENCE TO RELATED APPLICATIONS

This application claims the benefit of U.S. Provisional Application No. 62/891,497, filed Aug. 26, 2019, which is incorporated herein by reference in its entirety.

### STATEMENT OF GOVERNMENTAL INTEREST

This invention was made with government support under grant nos. CA210985, Al122382, and CA152813, awarded by the National Institutes of Health. The government 10 has certain rights in the invention.

### INCORPORATION-BY-REFERENCE OF MATERIAL SUBMITTED ELECTRONICALLY

This application contains a sequence listing. It has been submitted electronically via EFS-Web as an ASCII text file entitled “P15799-02_ST25.txt.” The sequence listing is 1,271 bytes in size, and was created on Aug. 21, 2020. It is hereby incorporated by reference in its entirety.

### FIELD OF THE INVENTION

The present invention relates to the field of protein post-translational modification. More specifically, the present invention provides compositions and methods useful for identifying O-linked glycosylation sites in proteins.

### BACKGROUND OF THE INVENTION

Over decades of biomedical investigations, it was found that one of the most distinctive features of cancers is the expression of Tn antigen (Tn), which is an N-acetylgalactosamine (GalNAc) attached to protein Ser/Thr residues via an O-linked glycosidic linkage¹. A variant of Tn is STn, which has an addition of sialic acid monosaccharide¹. Tn establishes its nature as a pan-carcinoma antigen by finding its expression in 10-90% of solid tumors including lung, prostate, breast, colon, pancreas, gastric, stomach, ovary, cervix, bladder¹⁻³. In sharp contrast, the expression of Tn in adult tissue is rare⁴, making it an attractive target for anti-cancer applications. For instance, Slovin et al. report a Phase I clinical trial using a vaccine consisting of synthetic Tn on a carrier protein for prostate cancers. Studies explore the potential of Tn for early diagnostics⁶⁻⁸ and prognostics of cancers⁹⁻¹¹. To treat cancers, Posey et al. report the development of engineered CAR-T cells that target Tn on mucin protein MUC1 (MUC1-Tn) for killing cancer cells¹². Also, a Phase I clinical trial using MUC1-Tn specific CAR-T cells started for treating patients with head and neck cancer^{13,14}. Despite a noteworthy link between Tn and cancers, the underlying mechanism causing the expression of Tn in cancers is not entirely clear. It may involve glycosyltransferase C1GalT1 and its chaperone C1GalT1C1 also called Cosmc¹⁵. Defective mutation in Cosmc is reported to affect the function of C1GalT1 for elongating Tn to normal O-glycan structures^{15,16}. Furthermore, Tn is involved in IgA nephropathy (IgAN, also known as Berger's disease) that is the most common glomerular disease in the world^{3,17,18}. A large percentage of patients with IgAN progress to kidney failure, also called end-stage renal disease (ESRD)^{3,17}. The cause of IgAN may involve the expression of Tn and STn on hinge region of IgA1³.

Although Tn is structurally simple, identification of its glycosylation sites and the carrier proteins in the complex samples is highly challenging due to the lack of suitable technology. Limited information regarding Tn-glycosylation sites and carrier proteins hamper the understanding of the role of Tn in cancer biology and the development of new strategies targeting cancers. Current methods for mapping Tn-glycosylation sites include the use of VVA lectin or hydrazide chemistry for the enrichment of Tn-glycopeptides, followed by LC-MS/MS for site localization^{19d20}. Jurkat T cells expressing Tn and STn, due to the mutation in Cosmc, are often used as a model system to evaluate the effectiveness of methods. Using VVA lectin chromatography and ETD-MS2, Steentoft et al. identify 68 O-glycoproteins in Jurkat cells¹⁹. Zheng et al. use galactose oxidase to oxidize Tn followed by solid-phase capture using hydrazide chemistry and release of Tn-glycopeptides using methoxyamine²⁰. Subsequent analysis using HCD-MS2 identifies 96 O-glycoproteins in three experiments with 87 glycosylation sites being localized in the first experiment of Jurkat cells²⁰. The present inventors, however, anticipate that about a thousand Tn-glycosylation sites remain to be mapped in Jurkat cells because 1,295 O-linked glycosylation sites are mapped in CEM cells, a human T cell line, using a method named EXoO developed in previous study²¹. It appears that the development of a technology capable of large-scale mapping of Tn-glycosylation sites would be a significant advance in technology and cancer biology.

### SUMMARY OF THE INVENTION

The present invention is based, at least in part, on the development of a new technology named EXoO-Tn that tags Tn and maps its glycosylation sites in a large-scale. EXoO-Tn utilizes two highly specific enzymes in a one-pot reaction for concurrent tagging of Tn and mapping of its glycosylation sites. In particular embodiments, the first enzyme is glycosyltransferase C1GalT1, which catalyzes UDP-Gal to add a galactose to Tn. When isotopically-labeled UDP-Gal(¹³C6) is used, Gal(¹³C6)-Tn is formed. The Gal(¹³C6)-Tn has a unique mass tag distinguishable to endogenous Gal-GalNAc and other glycans. The second enzyme is an endoprotease named OpeRATOR, which cleaves at N-termini of Ser/Thr residues occupied by the Gal(¹³C6)-Tn to release site-containing Gal(¹³C6)-Tn-glycopeptides with the glycosylation sites positioning at the N-termini of peptide sequences. The two enzymes are synergistically integrated with the use of solid-phase for optimal removal of contaminants and efficient isolation of site-containing Gal(¹³C6)-Tn-glycopeptides. A proof of principle of EXoO-Tn was developed using a synthetic Tn-glycopeptide. The performance of EXoO-Tn was evaluated using Jurkat cells.

In one embodiment, the present invention provides a method for identifying O-linked glycosylation sites of Tn antigen in proteins comprising the steps of (a) digesting proteins present in a sample into peptides; (b) enriching for Tn-glycopeptides; (c) conjugating Tn-glycopeptides to solid phase; (d) labeling Tn using the glycosyltransferse enzyme C1GalT1 and a labeled uridine diphosphate galactose (UDP-Gal) substrate to produce labeled Tn-glycopeptides; (e) releasing the labeled Tn-glycopeptides from the solid-phase using an endopeptidase that cleaves peptides at the N-terminus of O-linked glycans at serine or threonine residues; and (f) mapping O-linked glycosylation sites of Tn antigen using liquid chromatography-mass spectrometry.

In certain embodiments, the proteins are present in a clinical sample obtained from a patient. In other embodiments, the proteins are present in a sample obtained from cell culture.

In a specific embodiment, the enrichment step (b) is performed using a lectin or hydrophilic interaction chromatography (HILIC). In another embodiment, the labeled UDP-Gal substrate comprises UDP-Gal(¹³C6), wherein Tn is converted to Gal(¹³C6)-Tn. In an alternative embodiment, the labeled UDP-Gal substrate comprises UDP-Gal(¹³C3), wherein Tn is converted to Gal(¹³C3)-Tn. In yet another embodiment, the labeled UDP-Gal substrate comprises UDP-Gal(¹³C1), wherein Tn is converted to Gal(¹³C1)-Tn.

In particular embodiments, prior to step (e), the labeled Tn-glycopeptides are treated with trifluoroacetic acid (TFA), a sialidase or a neuraminidase to remove sialic acid. In another embodiment, the digestion of step (a) is performed using trypsin. In other embodiments, steps (d) and (e) are performed simultaneously.

In a particular embodiment, a method for identifying O-linked glycosylation sites of Tn antigen in proteins comprises the steps of (a) digesting proteins present in a sample into peptides; (b)enriching for Tn-glycopeptides; (c) conjugating Tn-glycopeptides to solid-phase; (d) converting Tn to Gal(¹³C6)-Tn using the glycosyltransferse enzyme C1GalT1 and its substrate UDP-Gal(¹³C6) to produce Gal(¹³C6)-Tn-glycopeptides; (e) releasing Gal(¹³C6)-Tn-glycopeptides from the solid phase using an endopeptidase that cleaves peptides at the N-terminus of O-linked glycans at serine or threonine residues; and (f) mapping O-linked glycosylation sites of Tn antigen using liquid chromatography-mass spectrometry.

In another aspect, the present invention provides a kit. In a specific embodiment, a kit comprises (a) a glycosyltransferase enzyme C1GalT1; (b) a UDP-Gal substrate; and (c) an endopeptidase that cleaves peptides at the N-terminus of O-linked glycans at serine or threonine residues. In one embodiment, the UDP-Gal substrate is labeled or capable of being labeled. In another embodiment, the kit further comprises an enzyme for digesting proteins into peptides. In yet another embodiment, the kit further comprises a lectin or HILIC chromatography column for enriching Tn-glycopeptides. In a further embodiment, the kit also comprises a solid-phase for conjugating Tn-glylcopeptides. In another embodiment, the kit further comprises TFA, a sialidase or a neuraminidase.

### BRIEF DESCRIPTION OF THE FIGURES

FIG. 1. Strategy of EXoO-Tn for tagging of Tn and mapping its glycosylation site.

FIG. 2A-2B. Mapping Tn-glycosylation sites by integrating Tn-engineering and OpeRATOR digestion. FIG. 2A: OpeRATOR digestion of Gal- and Gal(¹³C6)-Tn-glycopeptide after Tn was tagged using C1GalT1 with UDP-Gal or UDP-Gal(¹³C6). Top left panel: the synthetic Tn-glycopeptide before treatments. Top middle panel: conversion of Tn to Gal-Tn using C1GalT1 and UDP-Gal. Bottom middle panel: OpeRATOR digestion of the Gal-Tn-glycopeptide generated in the top middle panel produced site-containing glycopeptide S(Gal-Tn)PSTPPTPSPSC-NH2 (SEQ ID NO:3) and peptide VPSTPPTP (SEQ ID NO:2). Top right panel: conversion of Tn to Gal(¹³C6)-Tn using C1GalT1 and UDP-Gal(¹³C6). Bottom right panel: OpeRATOR digestion of the Gal(¹³C6)-Tn-glycopeptide engineered in the top right panel yielded site-containing glycopeptide S(Gal(¹³C6)-Tn)PSTPPTPSPSC-NH2 (SEQ ID NO: 3) and peptide VPSTPPTP (SEQ ID NO:2). FIG. 2B: HCD-MS2 spectrum of site-containing Gal(¹³C6)-Tn-glycopeptide identified in Jurkat cells. A diagnostic oxonium ion at 372 m/z corresponding to fragmentation ion of Gal(¹³C6)-Tn was colored in purple.

FIG. 3. A Schematic workflow for identification of site-specific Tn-glycoproteome in Jurkat cells.

FIG. 4A-4E. Characteristics of site-specific Tn-glycoproteome in Jurkat cells. FIG. 4A: The overall intensity of oxonium ions at 204 and 372 m/z in the assigned PSMs. The overall intensity of oxonium ion at 372 m/z was 10-fold less than that of 204 m/z. FIG. 4B: Motif analysis revealed the conserved motif of Tn-glycosylation sites. FIG. 4C: GO analysis revealed cellular components for Tn-glycoproteome. FIG. 4D: Analysis of the relative position of Tn-glycosylation sites in protein sequences revealed that the frequency of Tn-glycosylation distributed evenly across protein sequences with lower frequency at protein termini. FIG. 4E: Comparison of O-linked glycosylation sites and glycoproteins identified in this and other studies^{19,20}.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

In certain embodiments, the method generally comprises the steps of (1) Digestion of protein to peptides; (2) Enrichment of glycopeptides; (3) Conjugation of enriched glycopeptides to solid-phase; (4) Conversion of Tn to Gal(¹³C6)-Tn or Gal(¹³C3)-Tn or Gal(¹³C1)-Tn; (5) Release of Gal(¹³C6)-Tn-glycopeptides or their variants, including Gal(¹³C3)-Tn-glycopeptides or Gal(¹³C1)-Tn-glycopeptides from solid-phase; and (6) Analysis of the Gal(¹³C6)-Tn-glycopeptides and their variants, including Gal(¹³C3)-Tn-glycopeptides and Gal(¹³C1)-Tn-glycopeptides.

One of ordinary skill in the art could utilize a range of conditions for any one of the method steps. For example, the proteins can be digested using different enzymes including, but not limited to, trypsin, Lys-C, Lys-N, CNBr, Arg-C, Asp-N, GluC, Chemotrypsin, Pepsin, Proteinase K, and Thermolysin. Combinations of multiple enzymes can be used to digest the proteins into peptides. The digestion reaction can be performed at room temperature or 37° C. or any temperature above 0° C.

As described in the Examples, the Tn-glycopeptides were enriched using either VVA (alternative name VVL) lectin or RAX cartridge. The Tn-glycopeptides from Jurkat cells and sera were enriched using VVA. The Tn-glycopeptides from pancreatic tissues were enriched using RAX cartridge. In another embodiment, the Tn-glycopeptides could be efficiently enriched using RAX cartridge after conversion of Tn to Gal(¹³C6)-Tn using C1GalT1 with UDP-Gal(¹³C6). Other enrichment methods can be used including, but not limited to, lectins, HILIC cartridge, RAX cartridge, MAX cartridge and the like.

The enriched glycopeptides can be conjugated to any solid-phase. In certain embodiments, the enriched Tn-glycopeptides are conjugated to beads through amine and aldehyde reduction.

The enzyme C1GalT1 can be used with its substrate UDP-Gal(¹³C6) to modify Tn to Gal(¹³C6)-Tn. In other embodiments, UDP-Gal(¹³C3) or Gal(¹³C1) can be used modify Tn to Gal(¹³C3)-Tn or Gal(¹³C1)-Tn, respectively.

Gal(¹³C6)-Tn-glycopeptides or their variants, including Gal(¹³C3)-Tn-glycopeptides or Gal(¹³C1)-Tn-glycopeptides, can be released from solid-phase using an O-protease that cleaves the peptide bond N-terminal to serine or threonine that is substituted with O-glycan, while non-O-glycosylated serine/threonine remains on the solid phase. In a particular embodiment, the endopeptidase is the enzyme OpeRATOR. In more specific embodiments, OpeRATOR and the enzyme SIALEXO can be used. SIALEXO is used to remove sialic acid to facilitate OpeRATOR digestion.

The enzyme reaction can be performed in wide range of buffers and temperatures. In an alternative embodiment, peptides can be treated with 0.1% TFA treatment at 75° C. for 1 hour to remove sialic acid. In other embodiments, neuraminidase also can be used to remove sialic acid.

In further embodiments, Gal(¹³C6)-Tn-glycopeptides and their variants, including Gal(¹³C3)-Tn-glycopeptides and Gal(¹³C1)-Tn-glycopeptides, can be analyzed using any LC-MS/MS instrumentation or a protein gel.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: EXoO-Tn Tag-n-Map the Tn Antigen in the Human Genome

Tn antigen (Tn), a single N-acetylgalactosamine (GalNAc) monosaccharide attached to protein Ser/Thr residues, is found on most solid tumors yet rarely detected in adult tissues, featuring it one of the most distinctive signatures of cancers. Although it is prevalent in cancers, Tn-glycosylation sites are not entirely clear owing to the lack of suitable technology. Knowing the Tn-glycosylation sites will spur the development of new vaccines, diagnostics, and therapeutics of cancers. Here, the present inventors report a novel technology named EXoO-Tn for large-scale mapping of Tn-glycosylation sites. EXoO-Tn utilizes glycosyltransferase C1GalT1 and, in particular embodiments, isotopically-labeled UDP-Gal(¹³C6) to tag and convert Tn to Gal(¹³C6)-Tn, which has a unique mass being distinguishable to other glycans. THIS exquisite Gal(¹³C6)-Tn structure is recognized by a human-gut-bacterial enzyme, called OpeRATOR, that specifically cleaves N-termini of the Gal(¹³C6)-Tn-occupied Ser/Thr residues to yield site-containing glycopeptides. The two enzymes C1GalT1 and OpeRATOR could be used concurrently in a one-pot reaction. The effectiveness of EXoO-Tn was benchmarked by analyzing Jurkat cells, where 947 Tn-glycosylation sites from 480 glycoproteins were mapped. Bioinformatic analysis of the identified site-specific Tn-glycoproteins revealed conserved motif, cellular localization, relative position in proteins, and a substantially large number of Tn-glycosylation sites identified by EXoO-Tn. Given the importance of Tn in diseases, EXoO-Tn is anticipated to have broad utilities in the translational and clinical studies.

Material and Methods

Tagging of Tn and mapping its glycosylation site using synthetic Tn-glycopeptide.

Synthetic Tn-glycopeptide VPSTPPTPS(α-GalNAc)PSTPPTPSPSC-NH2 (SEQ ID NO:1) IgA1 hinge peptide was purchased from Susses Research. In the workflow with sequential enzymatic treatments, five μg of glycopeptide in 50 mM Tris-HCl pH 7.4 was mixed with one μg recombinant human C1GalT1/C1GalT1C1 protein (R&D Systems, NM) in the presence of either 0.5 mM UDP-Gal (Sigma-Aldrich) or 0.5 mM UDP-Gal¹³C6 (Omicron Biochemicals, Inc., IN) at 37° C. for 16 hours. After incubation, half of each sample was subjected to digestion using five units of OpeRATOR (Genovis Inc, Cambridge, Mass.) at 37° C. for 16 hours. The glycopeptides were desalted using C18 ZipTip (Millipore Sigma), dried using speed-vac, and resuspended in 0.1% TFA. In the concurrent one-pot enzymatic treatment that was used in all experiments described below, enzymes including C1GalT1/C1GalT1C1, OpeRATOR, and substrate i.e., UDP-Gal or UDP-Gal¹³C6 were added at the same time using the amount as described in the above sequential enzymatic workflow and incubated at 37° C. for 16 hours before C18 desalting and LC-MS/MS analysis.

Extraction of site-containing Tn-glvcogpetides from Jurkat cells. Jurkat Clone E6-1 (NIH AIDS Reagent Program) were cultured and expanded in RPMI 1640 supplemented with 10% fetal bovine serum (FBS), 100 units of penicillin, and 100 μg of streptomycin. The cells were collected, washed three times in the ice-cold PBS and lysed in 8 M urea/500 mM ammonia bicarbonate. The cell lyse was sonicated and centrifuged at 16,000 g to remove particles. Protein concentration was determined using a protein BCA assay. Twenty milligrams of proteins were reduced in 5 mM DTT at 37° C. for 1 hour and alkylated in 10 mM iodoacetamide at room temperature (RT) for 40 min in the dark. The samples were then diluted five-fold using 100 mM ammonia bicarbonate buffer. Trypsin was added to the samples with an enzyme/protein ratio of 1/40 w/w. After incubation at 37° C. for 16 hours, lysine residues were guanidination-modified, and peptides were desalted using C18 cartridges (Waters, Milford, Mass.), as described in the previous study”. The peptides were dried using speed-vac, resuspended in PBS with α2-3,6,8 neuraminidase (New England Biolabs, Ipswich, Mass.), and incubated at 37° C. for 16 hours. Four-hundred microliters agarose bound Vicia Villosa Lectin (VVA) (50% slurry, Vector Laboratories, Burlingame, Calif.) were washed twice using water, added to peptides and incubated at RT for 16 hours with rotation. The VVA agarose was gently washed with 1×PBS for three times. Bound glycopeptides were eluted using 4 M urea/100 mM Tris-HCl pH 7.4/400 mM GaINAc (Sigma-Aldrich) at RT for 30 min with shaking. The eluted glycopeptides were desalted using C18 cartridge and conjugated to AminoLink resin (Pierce, Rockford, Ill.) as described previously²¹. Briefly, the pH of C18 elute containing glycopeptides was neutralized to approximately pH 7 using two volume of 10× PBS. The solution was mixed with resin (100 μg peptide/100 μl resin, 50% slurry) and 50 mM sodium cyanoborohydride (NaCNBH₃) at RT for a minimal of 4 hours or overnight with rotation. Unreacted groups on resin were blocked using 1M Tris-HCl buffer (pH 7.4) with 50 mM NaCNBH₃ at RT for 30 min with rotation. The resin was sequentially washed using 50% ACN, 1.5 M NaCl, and 50 mM Tris-HCl buffer (pH 7.4). To tag and release Tn-glycopeptides, a solution (50 μl) containing 10 μg of C1GalT1/C1GalT1C1, 0.5 mM UDP-Gal¹³C6, and 2000 units of OpeRATOR was added to the resin and incubated at 37° C. for 16 hours. The released glycopeptides in the solution were collected twice using 400 μl of 50 mM Tris-HCl buffer (pH 7.4). Glycopeptides in the collected solution were combined, desalted using C18 cartridge, dried using speed-vac, and resuspended in 0.1% TFA. The peptides were fractionated using HPLC and concatenated to eight fractions before LC-MS/MS analysis.

LC-MS/MS analysis. One microgram of glycopeptides was analyzed on a Fusion Lumos mass spectrometer with an EASY-nLC 1200 system or an LTQ Orbitrap Velos mass spectrometer (Thermo Fisher Scientific, Bremen, Germany). The mobile phase flow rate was 0.2 μl/min with 0.1% FA/3% acetonitrile in water (A) and 0.1% FA/90% acetonitrile (B). The gradient profile was set as follows: 6% B for 1 min, 6-30% B for 84 min, 30-60% B for 9 min, 60-90% B for 1 min, 90% B for 5 min and equilibrated in 50% B, flow rate was 0.5 μL/min for 10 min. MS analysis was performed using a spray voltage of 1.8 kV. Spectra (AGC target 4×10⁵ and maximum injection time 50 ms) were collected from 350 to 1800 m/z at a resolution of 60 K followed by data-dependent HCD MS/MS (at a resolution of 50 K, collision energy 36, AGC target of 2×10⁵ and maximum IT 250 ms) of the 15 most abundant ions using an isolation window of 0.7 m/z. Include charge state was 2-6. The fixed first mass was 110 m/z. Dynamic exclusion duration was 45 s.

Database search of site-containing Tn-glvcoegptides. A UniProt human protein database (71,326 entries, downloaded Oct. 19, 2017) was used to generate a peptide database with 26,067,074 non-redundant peptide entries using the method as described in the previous study²¹. Briefly, a randomized decoy database using The Trans-Proteomic Pipeline (TPP)²² was generated and concatenated with the target database. The concatenated database was digested with trypsin and then OpeRATOR in silico. Peptides with Ser or Thr residues and lengths from 6 to 46 amino acids were used. SEQUEST in Proteome Discoverer 2.2 (Thermo Fisher Scientific) was used to search with variable modification: oxidation (M), Gal¹³C6(1)HexNAc(1) (SiT), Hex(1)HexNAc(1) (SiT) and HexNAc (SiT) and static modification: carbamidomethylation (C) and guanidination (K). FDR was set at 1% using Percolator. Only MS/MS scans with oxonium ion at 204, and two of the other oxonium ions were kept. Assignments with XCorr score below one were removed. MS/MS spectra were manually studied and inspected using spectral viewer in Proteome Discoverer to identify the spectral feature and ensure the confidence of identification.

Bioinformatics. Software pLogo was used to reveal motif for Tn-glycosylation sites²³ surrounding by 15 amino acids in length with the central amino acids being the sites. The Database for Annotation, Visualization and Integrated Discovery (DAVID) and UniProt were used for Gene Ontology (GO) analysis²⁴. Python (version 2.7) is used to analyze the data and generate the figures, including the relative position of Tn-glycosylation sites in protein sequence, radar charts, unsupervised hierarchical clustering, and box plot.

Data Availability. The LC-MS/MS data have been deposited to the PRIDE partner repository²³ with the dataset identifier: project accession: PXD014390.

Results

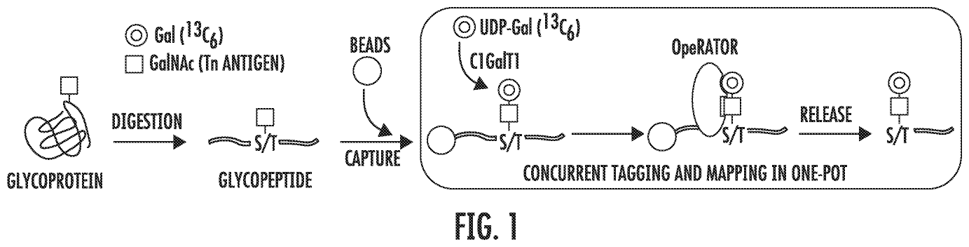

Principle of EXoO-Tn. EXoO-Tn includes six steps (FIG. 1). (i) Digestion: proteins extracted from samples are digested to peptides. Amino groups on the side chain of Lys residues are modified using guanidination on C18 cartridge. (ii) Enrichment: Tn-glycopeptides are enriched using VVA lectin. (iii) Conjugation: the enriched glycopeptides are conjugated to aldehyde-functionalized solid-phase through amino groups at the peptide N-termini. (iv) Tn-engineering: Tn is catalyzed to Gal(¹³C6)-Tn using C1GalT1/C1GalT1C1 and UDP-Gal(¹³C6). C1GalT1/C1GalT1C1 is specific to modify Tn. The Gal(¹³C6)-Tn has a unique mass that is distinguishable to endogenous Gal-GalNAc and other glycans in the samples. (v) Release: site-containing Gal(¹³C6)-Tn-glycopeptides are specifically released from solid-phase using OpeRATOR enzyme, which cleaves N-termini of Gal(¹³C6)-Tn-occupied Ser/Thr residues. (vi) Analysis: the released glycopeptides are analyzed using LC-MS/MS and software tools.

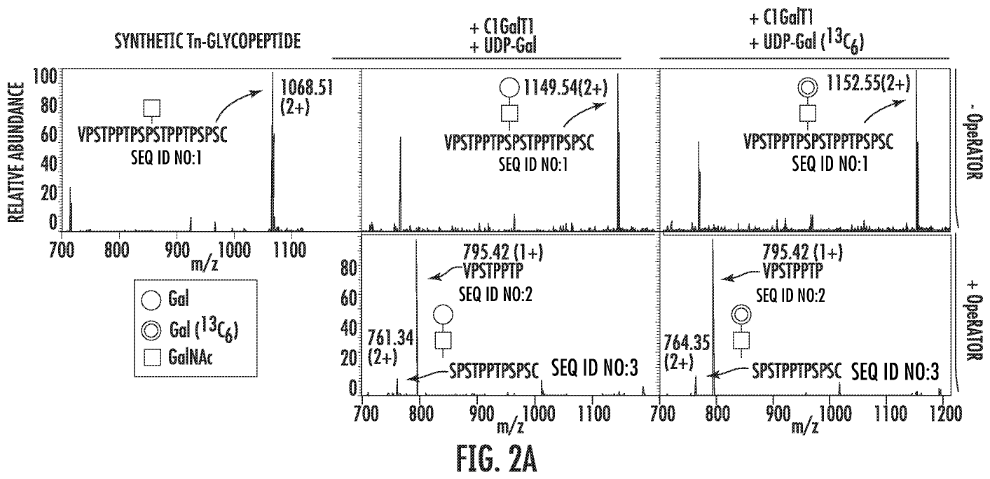

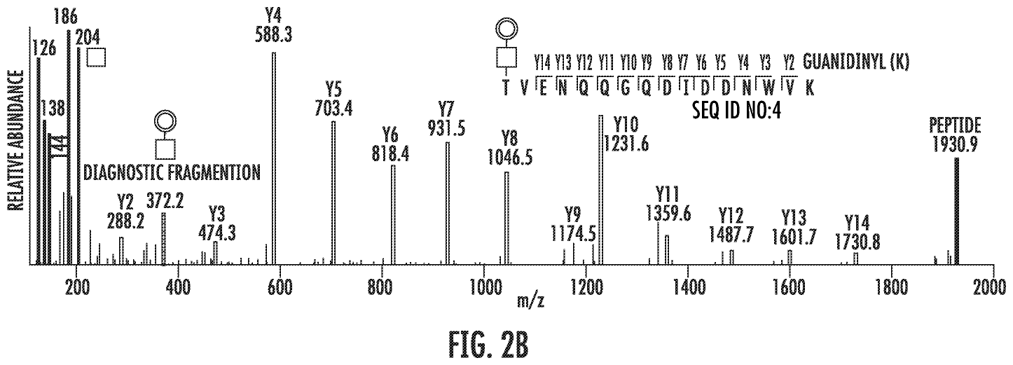

To show the feasibility of EXoO-Tn, a synthetic Tn-glycopeptide VPSTPPTPS(α-GalNAc)PSTPPTPSPSC-NH2 (SEQ ID NO:1) was used (FIG. 2A top left panel). The use of C1GalT1 and UDP-Gal converted Tn to Gal-Tn produced a charge+2 Gal-Tn-glycopeptide at 1149.54 m/z (FIG. 2A top middle panel), an increase of ˜162 Da corresponding to the mass of a galactose compared to its unmodified counterpart at 1068.51 m/z (FIG. 2A top left panel). The Gal-Tn-glycopeptide could be digested by OpeRATOR to yield site-containing glycopeptide S(Gal-Tn)PSTPPTPSPSC-NH2 (SEQ ID NO: 3) at 761.34 m/z and peptide VPSTPPTP (SEQ ID NO:2) at 795.42 m/z (FIG. 2A bottom middle panel). To distinguish the newly engineered Gal-Tn from endogenous Gal-GalNAc and other glycans, the UDP-Gal was substituted by an isotopically-labeled UDP-Gal(¹³C6). The Gal(¹³C6) has all six carbon molecules in galactose labeled with carbon-13 featuring an increment mass of 6 Da. The use of C1GalT1 and UDP-Gal(¹³C6) successfully converted Tn to Gal(¹³C6)-Tn with a unique mass tag of 371 and yielded a charge+2 Gal(¹³C6)-Tn-glycopeptide at 1152.55 m/z (FIG. 2A top right panel), which had an increase of ˜6 Da compared to its charge+2 Gal-Tn counterpart at 1149.54 m/z (FIG. 2A top middle panel). The site-containing glycopeptide S(Gal(¹³C6)-Tn)PSTPPTPSPSC-NH2 (SEQ ID NO:3) and peptide VPSTPPTP (SEQ ID NO:2) at 764.35 and 795.42 m/z, respectively, was generated after OpeRATOR digestion (FIG. 2A bottom right panel). The Gal(¹³C6)-Tn-glycopeptide had an increase of ˜6 Da compared to its Gal-Tn or endogenous Gal-GalNAc counterpart at 761.34 m/z (FIG. 2A bottom middle panel). Next, the MS/MS spectra of site-containing Gal(¹³C6)-Tn-glycopeptides were analyzed using HCD-MS2 to identify spectral feature for improvement of confidence of identification. As an illustration, an MS/MS spectrum of site-containing Gal(¹³C6)-Tn-glycopeptide from analysis of Jurkat cells was shown (FIG. 2B). A diagnostic oxonium ion generated by HCD fragmentation was observed at 372 m/z for the Gal(¹³C6)-Tn (FIG. 2B). The presence of the diagnostic oxonium ion at 372 m/z was utilized in the data interpretation. The Gal(¹³C6)-Tn-glycosylation site was informed to be the Thr residue at the N-terminus of the identified peptide sequence (FIG. 2B). Other fragmentation ions in the MS/MS spectrum, including oxonium ions, peptide b- and y-ions, and peptide ion supported the identification of the glycopeptide (FIG. 2B). The analysis of glycopeptides demonstrated the key enzymatic steps in EXoO-Tn to distinguish Tn from Gal-GalNAc and other glycans by isotopic tagging using C1GalT1 and UDP-Gal(¹³C6), and map Tn-glycosylation sites using OpeRATOR and LC-MS/MS.

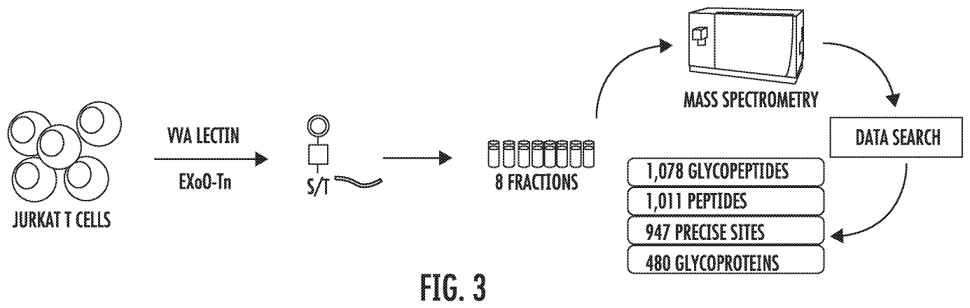

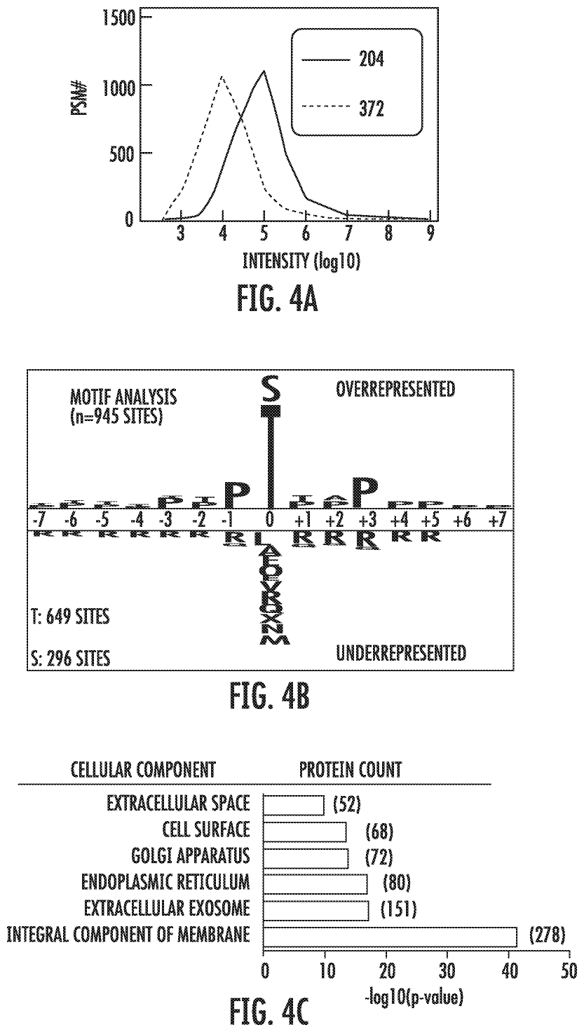

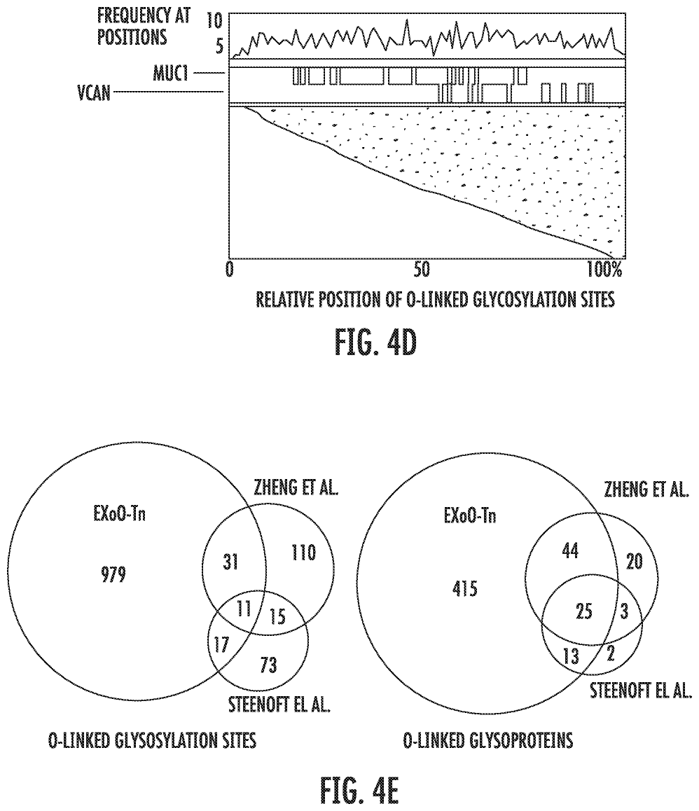

Mapping site-specific Tn-glycoproteome in Jurkat cells. Jurkat cells were analyzed to evaluate the performance of EXoO-Tn. With 1% FDR, 3,172 peptide-spectrum match (PSM) were assigned to 1,078 unique site-containing Gal(¹³C6)-Tn-glycopeptides that contained 1,011 unique peptide sequences (FIG. 3 and Supplementary Table 1 (data not shown, available on the bioRxiv website, https://doi.org/10.1101/84029)). From the peptide sequence, the present inventors mapped 947 Gal(¹³C6)-Tn-glycosylation sites from 480 glycoproteins (FIG. 3 and Supplementary Table 1 (data not shown)). The diagnostic oxonium ion at 372 m/z was detected in 96.4% of the assigned MS/MS spectra with an overall intensity being ten-fold lower than that at 204 m/z (FIG. 4A and Supplementary Table 1 (data not shown)). The detection of oxonium ion at 372 m/z in the assigned MS2 spectra supported the presence of Gal(¹³C6)-Tn in the identified glycopeptides (Supplementary Table 1 (data not shown)). It was observed that, among the assigned PSMs, approximately 89.2% glycopeptides were modified by a single Gal(¹³C6)-Tn composition while approximately 9.5 and 1.3% PSMs were modified by two or three Gal(¹³C6)-Tn compositions, respectively (Supplementary Table 1 (data not shown)).

Characterization of the site-specific Tn-glvcoproteome in Jurkat cells. Analysis of the glycosylation sites showed that hr and Ser accounted for approximately 68.7% and 31.3%, respectively. Motif analysis of ±7 amino acids surrounding 946 glycosylation sites found an overrepresentation of Pro residues at the +3 and −1 position (FIG. 4B). Two glycosylation sites residing close to the protein N-termini were not used in the motif analysis. Gene Ontology (GO) analysis of the identified glycoproteins found that integral component of membrane, extracellular exosome, endoplasmic reticulum (ER), Golgi apparatus, cell surface, and extracellular space were enriched for cellular component suggesting the presence of the identified glycoproteins in the secretory pathway and on the cell surface (FIG. 4C). Next, the relative position of the glycosylation sites in protein sequence was plotted and showed that proteins MUC1 and versican core protein (VCAN) had the highest number of glycosylation sites reaching 48 and 11, respectively (FIG. 4D middle panel). Besides, it was observed that the frequency of the glycosylation site was relatively even across protein sequences with lower frequency at protein termini (FIG. 4D top and bottom panels). Comparison of site-specific Tn-glycoproteome identified by EXoO-Tn to two other methods^{19,20} (Supplementary Table 2 and 3 (data not shown, available on the bioRxiv website, https://doi.org/10.1101/84029)) revealed that 888 Tn-glycosylation sites from 398 glycoproteins were exclusively identified using EXoO-Tn (FIG. 4E). Analysis of Jurkat cells established the effectiveness of EXoO-Tn to map the site-specific Tn-glycoproteome in the complex sample.

### DISCUSSION

A new technology EXoO-Tn has been developed for large-scale mapping Tn-glycosylation sites in a complex sample. EXoO-Tn has several advantages including (i) large-scale mapping of Tn-glycosylation sites in the complex sample; (ii) a tagging strategy for distinguishing engineered Tn from endogenous Gal-GalNAc and other glycans; (iii) concurrent tagging of Tn and release of site-containing Tn-glycopeptides from solid-phase in a one-pot fashion; (iv) applicable to analyze mucin-type O-linked glycoproteins; (v) no need of ETD for site localization.

C1GalT1 is a natural enzyme with specificity for extending O-GalNAc to core 1 Gal-GalNAc structure. OpeRATOR enzyme is utilized by bacteria to digest mucin glycoproteins in the gut with a specificity at N-termini of Gal-GalNAc occupied Serfrhr residues. The two enzymes work synergistically to render EXoO-Tn the specificity for mapping Tn-glycosylation sites. It is meritorious that Tn is tagged to have a unique mass and generate a diagnostic oxonium ion in the MS2 spectrum. The unique mass tag and diagnostic oxonium ion are useful to improve the confidence of identification. The use of solid-phase allows extensive washes that are essential to remove other peptides and contaminants while enables further enrichment of site-containing glycopeptides for LC-MS/MS analysis.

The present inventors mapped 947 Tn-glycosylation sites from almost 500 glycoproteins, a substantially large number of site-specific Tn-glycoproteome, which demonstrated the effectiveness of EXoO-Tn and supported that a large number of O-linked glycosylation sites could be mapped in cells. Some site-containing Tn-glycopeptides may be too long or too short to be detected using EXoO-Tn with trypsin digestion. Digestion of proteins using proteases with different specificities may further increase the identification number of glycosylation sites in EXoO-Tn methodology. Also, the identification of glycopeptides with two or three Gal(¹³C6)-Tn compositions suggests many more glycosylation sites in the peptide sequences supporting an even larger number of Tn-glycosylation sites in Jurkat cells. Characterization of glycosylation sites and glycoproteins identified in Jurkat cells revealed conserved features of protein O-linked glycosylation, including consensus motif, cellular localization, and distribution of the relative position of glycosylation sites across the protein sequences, a reminiscence of that seen in human kidney, serum, and T cells in the previous study². Given that Tn is prevalent in cancers and other diseases, EXoO-Tn is anticipated to have broad translational and clinical utilities.

### REFERENCES

1. Julien, S., Videira, P. A. & Delannoy, P. Sialyl-tn in cancer: (how) did we miss the target? Biomolecules 2, 435-466 (2012).
2. Munkley, J. The Role of Sialyl-Tn in Cancer. Internationaljournal of molecular sciences 17, 275 (2016).
3. Ju, T. et al. Tn and sialyl-Tn antigens, aberrant O-glycomics as human disease markers. Proteomics. Clinical applications 7, 618-631 (2013).
4. Kudelka, M. R., Ju, T., Heimburg-Molinaro, J. & Cummings, R. D. Simple sugars to complex disease—mucin-type O-glycans in cancer. Advances in cancer research 126, 53-135 (2015).
5. Slovin, S. F. et al. Fully synthetic carbohydrate-based vaccines in biochemically relapsed prostate cancer: clinical trial results with alpha-N-acetylgalactosamine-O-serine/threonine conjugate vaccine. Journal of clinical oncology: official journal of the American Society of Clinical Oncology 21, 4292-4298 (2003).
6. Itzkowitz, S. H., Bloom, E. J., Lau, T. S. & Kim, Y. S. Mucin associated Tn and sialosyl-Tn antigen expression in colorectal polyps. Gut 33, 518-523 (1992).
7. Inoue, M., Ton, S. M., Ogawa, H. & Tanizawa, O. Expression of Tn and sialyl-Tn antigens in tumor tissues of the ovary. American journal of clinical pathology 96, 711-716 (1991).
8. Wei, H. et al. Glycoprotein screening in colorectal cancer based on differentially expressed Tn antigen. Oncology reports 36, 1313-1324 (2016).
9. Nakagoe, T. et al. Prognostic value of circulating sialyl Tn antigen in colorectal cancer patients. Anticancer research 20, 3863-3869 (2000).
10. Tsuchiya, A. et al. Prognostic Relevance of Tn Expression in Breast Cancer. Breast cancer 6, 175-180 (1999).
11. Ohno, S. et al. Expression of Tn and sialyl-Tn antigens in endometrial cancer: its relationship with tumor-produced cyclooxygenase-2, tumor-infiltrated lymphocytes and patient prognosis. Anticancer research 26, 4047-4053 (2006).
12. Posey, A. D., Jr. et al. Engineered CAR T Cells Targeting the Cancer-Associated Tn-Glycoform of the Membrane Mucin MUC1 Control Adenocarcinoma. Immunity 44, 1444-1454 (2016).
13. Wilkie, S. et al. Retargeting of human T cells to tumor-associated MUC1: the evolution of a chimeric antigen receptor. Journal of immunology 180, 4901-4909 (2008).
14. Maher, J. et al. Targeting of Tumor-Associated Glycoforms of MUC1 with CAR T Cells. Immunity 45, 945-946 (2016).
15. Ju, T. et al. Human tumor antigens Tn and sialyl Tn arise from mutations in Cosmc. Cancer research 68, 1636-1646 (2008).
16. Hofmann, B. T. et al. COSMC knockdown mediated aberrant O-glycosylation promotes oncogenic properties in pancreatic cancer. Molecular cancer 14, 109 (2015).
17. Moran, S. & Cattran, D. C. IgA nephropathy: un update. Minerva medica (2019).
18. Berger, J. & Hinglais, N. [Intercapillary deposits of IgA-IgG]. Journal d'urologie et de nephrologie 74, 694-695 (1968).
19. Steentoft, C. et al. Mining the O-glycoproteome using zinc-finger nuclease-glycoengineered SimpleCell lines. Nature methods 8, 977-982 (2011).
20. Zheng, J., Xiao, H. & Wu, R. Specific Identification of Glycoproteins Bearing the Tn Antigen in Human Cells. Angewandte Chemie 56, 7107-7111 (2017).
21. Yang, W., Ao, M., Hu, Y., Li, Q. K. & Zhang, H. Mapping the O-glycoproteome using site-specific extraction of O-linked glycopeptides (EXoO). Mol Syt Biol 14, e8486 (2018).
22. Deutsch, E. W. et al. Trans-Proteomic Pipeline, a standardized data processing pipeline for large-scale reproducible proteomics informatics. Proteomics. Clinical applications 9, 745-754 (2015).
23. O'Shea, J. P. et al. pLogo: a probabilistic approach to visualizing sequence motifs. Nature methods 10, 1211-1212 (2013).
24. Huang da, W., Sherman, B. T. & Lempicki, R. A. Systematic and integrative analysis of large gene lists using DAVID bioinformatics resources. Nat Protoc 4, 44-57 (2009).
25. Vizcaino, J. A. et al. 2016 update of the PRIDE database and its related tools. Nucleic acids research 44, D447-456 (2016).
26. Weiss, A., Wiskocil, R. L. & Stobo, J. D. The role of T3 surface molecules in the activation of human T cells: a two-stimulus requirement for IL 2 production reflects events occurring at a pre-translational level. Journal of immunology 133, 123-128 (1984).

### Example 2: EXoO-Tn Protocol. For Cell/Tissue Lysis

Materials

Urea (solid) (Sigma U0631-1KG)

5M NaCl (Santa Cruz Biotechnology, sc-295833)

1M Tris HCl pH 8.0 (Ambion AM9855G)

Sequencing grade modified Trypsin (Promega; (V51 IX) Waters tC18 SepPak, 100 mg for desalting of 1-3 mg peptides, 1-3% binding capacity

(Waters; WAT054925)

C1GalT1/C1GalT1C1 (R&D Systems)

UDP-Gal(¹³C6) (Omicron Biochemicals, Inc.)

OpeRATOR also called OgpA (Genovis)

SialEXO (Genovis)

Trizma hydrochloride solution; pH 7.4, 1M

DTT (Thermo Fisher Pierce; cat#20291)

IAA (Sigma; cat# A3221-1OVL or Sigma; cat# Il149-5G)

Reagent Setup

8M urea buffer. Fill a 15 ml tube with urea powder to 4.8 g. Add 2 ml 1M TrisHCl pH 8. Fill H2O to the tube to 10 ml mark. Warm the tube in hand to properly dissolve the urea in the buffer. Make fresh before use.

1M DTT (WM 154.25. 200×) (Thermo Fisher Pierce: cat#20291). Weigh 7.7125 mg. Add 50ul H2O, to make 50ul of solution, make fresh before use.

500 mM IAA STOCK (WM 184.96, 50×) (Sigma: A3221-1OVL or Sigma: I1149-5G). Weigh 9.24 mg and add 50ul H2O, to make 50ul of solution, make fresh before use.

60% ACN/0.1% TFA. Mix 60 ml of ACN, 40 ml of H2O and 200ul of 50% TFA.

50% TFA. Mix 1 ml H2O and 1 ml TFA.

0.1% TFA. Mix 499 ml H2O and 1 ml of 50% TFA.

GUANIDINATION BUFFER. Mix equal volumes of 2.85M aqueous ammonia hydroxide, 0.1% TFA, and 0.6M O-methylisourea, final pH 10.5 (1:1:1).

1M SODIUM CYANOBOROHYDRIDE (WM 63. 20×). Weigh 63g and dissolve in 1 ml H2O.

Lectin elution buffer. 400 mM GalNAc/4M urea/200 mM TrisHCl pH 8 Make 8M urea in 200 mM TrisHCl pH 8. N-Acetyl-D-galactosamine 5 g sigma A2795-5G dissolve in 28.25 ml H2O, aliquoted and store in −20° C. Mix equal volume of 800 mM GalNAc and 8M urea/200 mM TrisHCl pH 8.

C18 DESALTING. Condition C18 cartridge using 60% ACN/0.1% TFA ×3 times, 0.1% TFA×3 times, load sample and let sample slowly pass through, wash with 0.1% TFA ×3 times, and finally elute in 400ul 60% ACN/0.1% TFA for using C18 with 100 mg bedding material.

DESALTING and GUANIDINATION on C18 CARTRIDGES. Peptides on C18 cartridge are washed with 0.1% TFA ×3 times and washed with guanidination buffer ×3 (keep enough guanidination buffer in the C18 cartridge to cover the C18 bedding material, seal the cartridge on top and bottom) and place the cartridges in a 65° C. incubator for 20 mins.

The cartridge is then transferred to 4° C. for 5 min to cool down. The cartridge is then wash with 0.1% TFA ×4 times and elute in 400ul 60% ACN/0.1% TFA for using C18 with 100 mg bedding material.

1.5M NaCl. Mix 75 ml 5M NaCl and 175 ml H2O.

100 mM TrisHCl pH 7.4. Mix 5 ml TrisHCl pH 7.4 and 45 ml H2O.

SialEXO. (Genovis Inc. cat# G2-OP1-020) add 50ul H2O to powder in the tube from the manufacture.

OpeRATOR. (Genovis Inc. cat# G2-OP1-020) add 50ul H2O to powder in the tube from the manufacture

C1 Gal1/C1GalT1C1. R&D Systems, cat#8659-GT-020

Procedure

1. Lysis of cells and tissue:
a. Place the sample on ice.
b. Weight the sample, write down the weight of sample.
c. Mix 8M urea buffer with cells with a 3:1 ratio.
d. Sonicate 3 time to dissolve all cell pellet in the buffer. After sonication, check the cell lysis solution to become complete aqueous solution.
e. Aliquot to 1.5 ml tube and centrifuge with high speed to remove undissolved particle.
f. Using BCA to determine protein amount.
g. Sample lyse can be stored in −80° C.

For lysis of tissue: cut the tissue into small piece using a scalpel on a glass slide.

Transfer the small pieces of tissue to 1.5 ml tubes using pipetting. Use minimal 8M urea buffer to collect the remaining tissue on the glass and transfer to the same 1.5 ml tube.

Pause Point

2. Reduce denatured proteins with DTT at final concentration of 5 mM at 37° C. for 1 h (1:200 dilution of 1M DTT).

3. Alkylate proteins with IAA at final concentration of 10 mM for 45 min at 25° C. or room temperature in the dark (1:50 dilution of 500 mM IAA stock).

4. Dilute sample at least 5 times to decrease urea concentration below 2 M with 100 mM TrisHCl pH 8.

5. Add Trypsin (Promega) in an enzyme to substrate ratio of 1:40 for ovemight (ca. 14-16 h) digestion at RT. Trypsin stock is at −0.5ug/uL-for 1 mg protein, add 50ul trypsin.

6. Add 50% TFA to acidify samples with a final concentration of 1% TFA. Check pH<3 using a pH paper.

7. There may be some precipitation after acidification of samples. Centrifuge the samples for 15 min using highest speed on a bench top centrifuge and transfer supernatant to new tubes. The digested samples can be stored in −20° C.

Pause Point

8. Adjust temperature of a heat incubator to 65° C.

9. Samples are desalted and guanidinated on C18 cartridges.

10. Elute the samples in 60% ACN/0.1% TFA. Nanodrop can be used to estimate the recovery of the peptides.

11. Dry the samples in a speed-vac. The dried sample can be stored in −20° C.

Pause Point

12. Thoroughly suspend peptides in PBS, centrifuge the sample for 10 mins with 15,000 g to remove particles, transfer the supernatant to new tubes, keep the pellet in −20 C, and add neuraminidase SIAEXO, 1U per lug peptide samples.

13. Incubate the samples at 37° C. overnight.

14. Take 200ul of VVA-agarose beads per 1 mg peptides and wash with H2O for twice, after the final wash, remove solution as much as possible.

15. Mix the samples with beads, add 100 mM CaCl₂), 100 mM MgCl2, and 100 mM MnCl2 to a final concentration of 1 mM. Rotate overnight at RT or 4° C.

16. Transfer sample to Pierce centrifuge filter columns and centrifuge to separate supernatant and beads. Do not need to wash the beads since lectin-glycopeptide interaction is found to be very week.

17. Use 200ul lectin elution buffer to transfer beads to new 1.5 ml tubes, use another 200ul to transfer remaining beads to the new 1.5 ml tube combining with the previous elution together 400ul. Strong vortex for 30 mins at RT.

18. Centrifuge down the beads and collect the elution, add 400ul PBS to beads, vortex and collect the PBS to combine with the elution to become 800ul. Centrifuge the 800ul elution to remove remaining beads.

19. Acidify the elution by adding 50% TFA to a final concentration of 1% TFA. The peptides are desalted using C18 cartridges. The amount of peptide in the C18 elute can be estimated using Nanodrop.

20. Neutralize the C18 elute using 3 fold volume of 10× PBS. Check pH about 7 using pH paper.

21. Take AminoLink beads, lug peptide to 1 ul beads. Wash beads with H2O twice, mix beads with samples.

22. Make 1 M sodium cyanoborohydride (WM 63, 20×) using H2O, add to the solution containing sample and beads. Final concentration of sodium cyanoborohydride is 50 mM, rotate at least 4 hours or overnight at RT.

23. Blocking. Transfer solution containing samples and beads to centrifuge filter column and centrifuge to remove supernatant. Seal bottom with plastic blocker. Add 700ul 1M TrisHCl 7.4 to beads and add 35ul 1M sodium cyanoborohydride (final concentration of 50 mM), mix well, rotate at least 30 mins at RT.

24. Wash the beads with 650ul of 60% ACN/0.1% TFA ×4 times, 1.5M NaCl ×4 times, and 100 mM TrisHCl pH 7.4 ×4 times. Vortex for 1 min for each wash step.

25. Transfer all the beads to a new 1.5 ml tube using 2 times of 400ul of 100 mM TrisHCl pH 7.4. Centrifuge down the beads, wait 10 mins let all beads settle. Remove supernatant to the level of upper line of beads.

26. Add 1 ul 5 mM UDP-Gal(¹³C6), 2ug of C1GalT1/C1GalT1C1 per 100ug peptides, and 100U OpeRATOR per 100ug peptides. Mix well the solution by pipetting. Do not vortex otherwise beads may retain on the wall of tubes that may decrease yield of glycopeptides. Incubate 37° C. overnight.

27. Centrifuge and collect supernatant. Add 400ul 100 mM TrisHCl pH 7.4 to beads to recovery the remaining glycopeptides, vortex for 2 mins, centrifuge, and collect the supernatant. Repeat this step once and combine all supernatant together.

28. Centrifuge, let sit 5 mins to allow the beads to settle. Transfer supernatant to a new tube. Repeat this step once make sure that no visible beads present in the solution.

29. Samples are acidified using 50% TFA and desalted using C18 cartridge. Amount of peptides in the C18 elute can be estimated using Nanodrop.

30. Dry the sample using speed-vac and thoroughly re-suspend in 0.1% TFA. Determine the peptide concentration using Nanodrop, peptides can be stored in −20° C.

LC-MS/MS Analysis

One microgram of glycopeptides was analyzed on a Fusion Lumos mass spectrometer with an EASY-nLC 1200 system or an LTQ Orbitrap Velos mass spectrometer (Thermo Fisher Scientific, Bremen, Germany). Alternatively, the sample can be analyzed using other mass spectrometry.

Data Analysis

The mass spectrometry raw file can be analyzed using SEQUEST in Proteome Discoverer software.

### Example 3: Identification of Tn-Glycosylated Markers in Cancer

EXoO-Tn was performed on sera from individuals with pancreatic cancer. The method identified several Tn-glycosylated proteins including, but not limited to, Tn-glycosylated Kininogen-1 (KNG1), Clusterin (CLU) and Complement Factor H-Related 5 (CFHR5). Accordingly, Tn-glycosylated KNG1, CLU and CFHR5 can be used in methods for diagnosing and/or prognosing pancreatic cancer.

1. A method for identifying O-linked glycosylation sites of Tn antigen in proteins comprising the steps of:
(a) digesting proteins present in a sample into peptides;
(b) enriching for Tn-glycopeptides;
(c) conjugating Tn-glycopeptides to solid phase;
(d) labeling Tn using the glycosyltransferse enzyme C1GalT1 and a labeled uridine diphosphate galactose (UDP-Gal) substrate to produce labeled Tn-glycopeptides;
(e) releasing the labeled Tn-glycopeptides from the solid-phase using an endopeptidase that cleaves peptides at the N-terminus of O-linked glycans at serine or threonine residues; and
(f) mapping O-linked glycosylation sites of Tn antigen using liquid chromatography-mass spectrometry.

2. The method of claim 1, wherein the proteins are present in a clinical sample obtained from a patient.

3. The method of claim 1, wherein the proteins are present in a sample obtained from cell culture.

4. The method of claim 1, wherein the enrichment step (b) is performed using a lectin or hydrophilic interaction chromatography (HILIC).

5. The method of claim 1, wherein the labeled UDP-Gal substrate comprises UDP-Gal(¹³C6), wherein Tn is converted to Gal(¹³C6)-Tn.

6. The method of claim 1, wherein the labeled UDP-Gal substrate comprises UDP-Gal(¹³C3), wherein Tn is converted to Gal(¹³C3)-Tn.

7. The method of claim 1, wherein the labeled UDP-Gal substrate comprises UDP-Gal(¹³C1), wherein Tn is converted to Gal(¹³C1)-Tn.

8. The method of claim 1, wherein prior to step (e), the labeled Tn-glycopeptides are treated with trifluoroacetic acid (TFA), a sialidase or a neuraminidase to remove sialic acid.

9. The method of claim 1, wherein the digestion of step (α) is performed using trypsin.

10. The method of claim 1, wherein steps (d) and (e) are performed simultaneously.

11. A method for identifying O-linked glycosylation sites of Tn antigen in proteins comprising the steps of:
(a) digesting proteins present in a sample into peptides;
(b) enriching for Tn-glycopeptides;
(c) conjugating Tn-glycopeptides to solid-phase;
(d) converting Tn to Gal(¹³C6)-Tn using the glycosyltransferse enzyme C1GalT1 and its substrate UDP-Gal(¹³C6) to produce Gal(¹³C6)-Tn-glycopeptides;
(e) releasing Gal(¹³C6)-Tn-glycopeptides from the solid phase using an endopeptidase that cleaves peptides at the N-terminus of O-linked glycans at serine or threonine residues; and
(f) mapping O-linked glycosylation sites of Tn antigen using liquid chromatography-mass spectrometry.

12. A kit comprising:
(a) a glycosyltransferase enzyme C1GalT1;
(b) a UDP-Gal substrate; and
(c) an endopeptidase that cleaves peptides at the N-terminus of O-linked glycans at serine or threonine residues.

13. The kit of claim 12, wherein the UDP-Gal substrate is labeled or capable of being labeled.

14. The kit of claim 12, further comprising an enzyme for digesting proteins into peptides

15. The kit of claim 12, further comprising a lectin or HILIC chromatography column for enriching Tn-glycopeptides

16. The kit of claim 12, further comprising a solid-phase for conjugating Tn-glylcopeptides;

17. The kit of claim 12, further comprising TFA, a sialidase or a neuraminidase.
